(19) United States
(12) Patent Application Publication
(10) Pub. No.: US 2005/0158702 A1
(43) Pub. Date: Jul. 21, 2005

(54) Cellular arrays comprising encoded cells

(76) Inventors: John R. Stuelpnagel, ENCINITAS, CA (US); Mark S. Chee, Del Mar, CA (US)

Correspondence Address: DORSEY & WHITNEY LLP;INTELLECTUAL PROPERTY DEPARTMENT, 4 EMBARCADERO CENTER, SUITE 3400, SAN FRANCISCO, CA, 94111, US

(21) Appl. No.: 10/363,240

(22) PCT Filed: Sep. 5, 2001
(86) PCT No.: PCT/US01/27510

Related U.S. Application Data
(62) Division of application No. 60/230,007, filed on Sep. 5, 2000.

(51) Int. Cl. C12Q 1/00; G01N 33/53; G01N 33/567; C12M 1/34

(52) U.S. Cl. 435/4; 435/287.1; 435/7.2

(57) ABSTRACT

A biosensor, sensor array, sensing method and sensing apparatus are provided in which individual cells or randomly mixed populations of cells, having unique response characteristics to chemical and biological materials, are deployed in a plurality of discrete sites on a substrate. In a preferred embodiment, the discrete sites comprise microwells formed at the distal end of individual fibers within a fiber optic array. The biosensor array utilizes an optically interrogatable encoding scheme for determining the identity and location of each cell type in the array and provides for simultaneous measurements of large number of individual cell respnses to target analyses. The sensing method utilizes the unique ability of cell populations to respond to biologically significant compounds in a characteristic and detectable manner. The biosensor array and measurement method may be employed in the study of biologically active materials in situ environmental monitoring, monitoring of a variety of bioprocesses, and for high throughput screening of large combinatorial chemical libraries.

The present application claims the benefit of U.S. Application Ser. No. 60/230,007, which is expressly incorporated herein by reference.

### FIELD OF THE INVENTION

The present invention is directed to biosensors, biosensor arrays, sensing apparatus and sensing methods which employ cells and mixed populations of cells, particularly encoded cells, for analysis of chemical and biological materials on cellular processes.

### BACKGROUND OF THE INVENTION

It is generally recognized that important technical advances in chemistry, biology and medicine benefit from the ability to perform microanalysis of samples in minute quantities. However, making analytical measurements on minute quantities has long been a challenge due to difficulties encountered with small volume sample handling, isolation of analyses, and micro-analysis of single-cell physiology.

Nanoliter, picoliter, and femtoliter volume studies have been explored in a range of applications involving in vitro and in vivo cellular investigations [R. M. Wightman, et al., Proc. Natl. Acad. Sci. U.S.A. 88: 10754 (1991); R. H. Chow, et al. Nature 356: 60 (1992); T. K. Chen, et al. Anal. Chem. 66: 3031 (1994); S. E. Zerby, et al., Neurochem. 66: 651 (1996); P. A. Garis, et al. J. Neurosci. 14: 6084 (1994); G. Chen, et al., J. Neurosci. 15: 7747 (1995)], electrochemistry [R. A. Clark, et al., Anal. Chem. 69(2): 259 (1997)], matrix assisted laser desorption-ionization mass spectrometry [S. Jespersen, et al., Rapid Commun. Mass Spectrom. 8: 581 (1994)], micro-column liquid chromatography [I. A. Holland, et al., Anal. Chem. 67: 3275 (1995); M. D. Oates, et al., Anal. Chem. 62: 1573 (1990)], micro-titration [M. Gratzl. et al Anal. Chem. 65: 2085 (1993); C. Yi, et al., Anal. Chem. 66: 1976 (1994)], and capillary electrophoresis [M. Jansson, et al., J. Chromatogr. 626: 310 (1992); P. Beyer Hietpas, et al. J. Liq. Chromatogr. 18: 3557 (1995)].

Clark, et al. [Anal. Chem. 69(2): 259 (1997)] has disclosed a method for fabricating picoliter microvials for electrochemical microanalysis using conventional photolithographic masking and photoresist techniques to transfer mold polystyrene microvials on silicon wafer templates. These microvials typically exhibit non-uniformity in size and shape due to the difficulty in controlling the re-etching of the molding surface and the transfer molding process.

Park, et al. [Science 276: 1401 (1997)] has disclosed a modified lithographic method for producing arrays of nanometer-sized holes using polystyrene-polybutadiene, ordered, diblock copolymers as masks in reactive ion etching of silicon nitride. This multi-step method is capable of producing arrays of picoliter-sized holes which are typically 20 nanometers in diameter and 20 nanometers deep with a spacing of 40 nanometers. Hole densities of up to 10¹¹ holes/cm² are disclosed. The range of sizes and spacings of the holes produced by this method is limited by the size of the copolymer microdomains. Uniformity of hole size and spacing is difficult to maintain with this method due to difficulties in controlling the etching method employed to form the holes.

Deutsch, et al. [Cytometry 16: 214 (1994)] have disclosed a porous electroplated nickel microarray comprised of micron-sized conical holes in blackened nickel plate. Hole sizes range from a 7 um upper diameter to a 3 um lower diameter with an 8 um depth. The array is used as a cell carrier for trapping individual cells while studying the responses of individual cells to changes in their microenvironment. In U.S. Pat. No. 4,772,540, Deutsch, et al., have also disclosed a method for making such an array using a combined photoresist and electroplating technique.

Corning Costar Corp. (Actor, Ma) produces a commercial microwell array for miniaturized assays under the trademark PixWell™. These arrays are made from microformed glass plates and comprise 40 um diameter by 20 um deep tapered wells with a well density of 4356 wells/cm².

Microwell arrays have particular utility in the study of living cells. In cell research, the measurement of responses of individual cells to changes or manipulations in their local environment is desirable. Any method or device designed for such studies must provide for the capability of maintaining cell viability, identifying the location of individual cells, and correlating response measurements with individual cells.

Due to the availability of viable fluorescent probes for intracellular studies, fluorescence measurements of living cells have significant utility in the study of cell functions. Thus fluorescence optical measurements are often utilized in cell studies where three generic methods of cell measurement are available, comprising bulk measurements of cell populations, dynamic measurements of cell populations or individual cells, and static measurements of individual cells.

The characteristics of an entire cell population as a whole can be studied with bulk measurements of sample volumes having a plurality of cells. This method is preferred where cell populations are very homogeneous. A generally recognized limitation of this method is the presence of background fluorescence which reduces the sensitivity of measurements and the inability of distinguishing differences or heterogeneity within a cell population.

Flow cytometry methods are often employed to reduce problems with background fluorescence which are encountered in bulk cell population measurements [M. R. Gauci, et al., Cytometry 25: 388 (1996); R. C. Boltz, et al., Cytometry 17: 128 (1994)]. In these methods, cell fluorescence emission is measured as cells are transported through an excitation light beam by a laminar flowing fluid. Flow cytometry methods may be combined with static methods for preliminary sorting and depositing of a small number of cells on a substrate for subsequent static cell measurements [U.S. Pat. No. 4,009,435 to Hogg, et al.; Kanz, et al., Cytometry 7: 491 (1986); Schildkraut, et al., J. Histochem Cytochem 27; 289 (1979)].

Gauci, et al., disclose a method where cell size, shape and volume is measured by light scattering and fluorescent dyes are utilized to determine protein content and total nucleic acid content of cells. This method further provides for counting and sizing various cells at a rate of approximately 100 cells per second.

Flow cytometry techniques are generally limited to short duration, single measurements of individual cells. Repetitive measurements on the same cell over time are not possible with this method since typical dwell times of a cell in the excitation light beam are typically a few microseconds. In addition, the low cumulative intensity from individual cell fluorescence emissions during such short measurement times reduces the precision and limits the reliability of such measurements.

Regnier, et al., [Trends in Anal. Chem. 14(4): 177 (1995)] discloses an invasive, electrophoretically mediated, microanalysis method for single cell analysis. The method utilizes a tapered microinjector at the injection end of a capillary electrophoresis column to pierce an individual cell membrane and withdraw a sample of cytoplasm. The method measures cell contents, one cell at a time. The method is generally limited to the detection of easily oxidized species.

Hogan, et al., [Trends in Anal. Chem. 12(1): 4 (1993)] discloses a microcolumn separation technique which may be utilized in combination with either a conventional gas chromatograph-mass spectrometer, micro thin layer chromatography or high pressure liquid manipulation of small cellular volumes. The sensitivity of the method is limited and may require pre-selection of target compounds for detection.

Static methods are generally the preferred method for measurements on individual cells. Measurement methods range from observing individual cells with a conventional optical microscope to employing laser scanning microscopes with computerized image analysis systems [see L. Hart, et al., Anal. QuanL Cyto/. Histol. 12: 127 (1990)]. Such methods typically require the attachment of individual cells to a substrate prior to actual measurements. Problems are typically encountered in attaching single cells or single layers of cells to substrates and in maintaining cells in a fixed location during analysis or manipulation of the cell microenvironment. Additionally, repetitive measurements on individual cells typically require physically indexing the location of individual cells and providing a mechanism for scanning each cell sequentially and returning to indexed cell locations for repeated analysis of individual cells.

Huang, et al., [Trends in Anal. Chem., 14(4) 158 (1995)] discloses a static electrochemical method and electrode for monitoring the biochemical environment of single cells. The method requires fabrication and manual positioning of a microelectrode reference and working electrode within the cell. The method has been used to detect insulin, nitric oxide and glucose inside single cells or external to the cells. The method is generally limited to the study of redox reactions within cells.

Ince, et al. [J. Immunol. Methods 128: 227 (1990)] disclose a closed chamber device for the study of single cells under controlled environments. This method employs a micro-perfusion chamber which is capable of creating extreme environmental conditions for cell studies. Individual cells are held in place by two glass coverslips as various solutions are passed through the chamber. One limitation of the method is the difficulty in eliminating entrapped gas bubbles which cause a high degree of autofluorescence and thus reduces the sensitivity of measurements due to background fluorescence.

In an attempt to overcome the limitations encountered with conventional static methods, Deutsch, et al., [Cytometry 16: 214 (1994)] and Weinreb and Deutsch, in U.S. Pat. Nos. 4,729,949, 5,272,081, 5,310,674, and 5,506,141, have disclosed an apparatus and method for repetitive optical measurements of individual cells within a cell population where the location of each cell is preserved during manipulation of the cell microenvironment.

A central feature of the apparatus disclosed by Deutsch, et al., is a cell carrier, comprising a two dimensional array of apertures or traps which are conical-shaped in order to trap and hold individual cells by applying suction. The cell carrier is typically fabricated by the combined electroplating-photoresist method disclosed in U.S. Pat. No. 4,772,540 to Deutsch, et al. The purpose of the cell carrier is to provide a means for maintaining the cells in fixed array locations while manipulating the cell environment. Individual cells are urged into cell carrier holes by suction and the wells are subsequently illuminated with a low-intensity beam of polarized light that reads back-emitted polarization and intensity. Measurements are compared when two different reagents are sequentially reacted with the cells. The method as disclosed requires two separate cell carriers for both a baseline control and analyte measurement.

The method and device of Deutsch, et al., have been employed by pathologists in diagnostic tests to determine the health and viability of cell samples taken from patients. The method and device have been applied to both cancer screening [Deutsch, et al., Cytometry 16: 214 (1994), Cytometry 23: 159 (1996), and European J. Cancer 32A (10): 1758 (1996)] and rheumatoid arthritis [Zurgil, et al., Isr. J. Med. Sci. 33: 273 (1997)] in which fluorescence polarization measurements are used to differentiate lymphocytes of malignant versus healthy cells based on changes in the internal viscosity and structuredness of the cytoplasmic matrix induced by exposure to tumor antigen and mitogens.

The method and device disclosed by Deutsch, et al., requires employment of a scanning table driven by three stepping motors and a computer control system for mapping, indexing and locating individual cells in the cell carrier. The use of such mechanical scanning methods introduces limitations in reproducibility and accuracy of measurements due to conventional mechanical problems encountered with backlash and reproducible positioning of individual cell locations for repeated measurements. In addition, mechanical scanning of the entire array prolongs the measurement time for each cell in the array.

The method disclosed by Deutsch, et al., is further limited by the use of fluorescence polarization measurements which have certain intrinsic limitations due to the significant influence of various optical system components on polarization as the fluorescence emission response is passed from the cell carrier to optical detectors. Birindelli, et al. [European J. Cancer 33(8): 1333 (1997)], has also identified limitations in this method due to fluctuations in electropolarisation values which require taking averages of at least three measurement scans for each condition so as to obtain reliable measurements. In addition, for cell studies, polarization measurements are generally limited to cell responses which produce sufficient changes in cytoplasm viscosity to produce a detectable change in polarization. Since not all cell responses are accompanied by detectable viscosity changes, the method is further limited to the cell activities which create such viscosity changes in the cytoplasm.

Zare. et al., [Science 267: 74 (1995); Biophotonics International, March-April, p 17 (1995)] discloses a biosensor system based on the response of living cells to complex biological materials fractionated by a microcolumn separation technique. Cells which were positioned on a glass cover slip were treated with a fluorescent probe and subsequently shown to be sensitive to a series of biological compounds including acetylcholine, bradykinin, and adenosine triphosphate as well as changes in intracellular calcium levels.

Yeung, et al. [Ace. Chem. Res. 27: 409 (1994)] has reviewed a number of methods for single cell response studies and has observed a significant variation and heterogeneity within cell populations based on analyte measurements. For example, the reference discloses a capillary electrophoresis method for exposing cells to biologically reactive compounds, extracting the intracellular fluid of individual cells produced in response to such compounds, and identifying analyses from migration times in the capillary column. Other fluorescence-based assays are also disclosed. Significant cell-to-cell variations and heterogeneity in individual cell responses within a cell population were observed which differences could provide a means for discriminating between biological and chemical compounds in contact with individual cells.

McConnell, et al. [Science, 257: 1906 (1992)], disclose a microphysiometer device known as the “Cytosensor” which uses a light addressable potentiometer sensor to measure the rate at which cells acidify their environment. This sensor acts as miniaturized pH electrode for monitoring cell responses which produce detectable changes in local pH. The disclosed device is limited to the measurement of proton excretions from cells and thus is only capable of detecting acidic cell responses to analyses.

U.S. Pat. No. 5,177,012 to Kim, et al., disclose a biosensor for the determination of glucose and fructose. The biosensor is produced by treating whole cells with an organic solvent and immobilizing the treated cells residue on a support to form a whole cell membrane which is applied to a pH electrode.

U.S. Pat. No. 5,690,894 to Pinkel, et al., discloses a biosensor which employs biological “binding partners”, materials such as nucleic acids, antibodies, proteins, lectins and other materials derived from cells, tissues, natural or genetically-engineered organisms. These agents are used in conjunction with a fiber optic array where each species of binding partners is uniquely addressed by a group of fibers within the fiber optic bundle which is coupled to an optical detector. The array was designed for screening of extensive arrays of biological binding partners.

While many of the prior art methods provide for the analysis of either single cells or populations of cells and some of these methods provide for monitoring cell responses to target analyses, none of the disclosed methods provides for employing large populations of monocultures or mixed populations of living cells for simultaneously monitoring the responses of individual cells to biological stimuli produced by chemical and biological analyses. Thus there is a need for a biosensor array and method which efficiently utilizes the ability of populations of living cells to respond to biologically significant compounds in a unique and detectable manner. Since the selectivity of living cells for such compounds has considerable value and utility in drug screening and analysis of complex biological fluids, a biosensor which makes use of the unique characteristics of living cell populations would offer distinct advantages in high throughput screening of combinatorial libraries where hundreds of thousands of candidate pharmaceutical compounds must be evaluated. In addition, such a sensor would be useful in monitoring bioprocesses and environmental pollution where the enhanced sensitivity of living cells to their environment can be exploited.

U.S. Ser. Nos. 09/033,462 and 09/260,963 describe arrays of cells that have been confined to microcavities, such as wells on the ends of a fiber optic bundle. These arrays may comprise either a single cell type or mixtures of cell types. For the latter, since the cells are randomly placed in the microcavities, these applications provide for encoding of cells through the use of membrane-binding fluorophores or the use of cells that have been genetically altered to produce different fluorophores, such as green fluorescent protein (GFP) variants.

However, a need exists for more robust and varied methods of encoding cell populations for the creation of cellular arrays.

### SUMMARY OF THE INVENTION

Accordingly, the invention provides a cellular array for detecting the response of individual cells to at least one analyte of interest. The array includes a substrate comprising a plurality of discrete sites and a plurality of cells dispersed at said discrete sites, wherein each cell is encoded with at least one exogenous binding partner.

In addition the invention provides a method of making a cellular array. The method includes associating a population of cells with a population of microspheres, such that each microsphere has at least one associated cell. Preferably the population includes a first subpopulation comprising a first cell type and at least a second subpopulation comprising a second cell type, wherein the first subpopulation comprises at least a first exogenous binding partner and the second subpopulation comprises at least a second exogenous binding partner. The method further includes distributing the population of microspheres onto discrete sites of a substrate, and identifying the location of at least the first and second exogenous binding partners.

In addition the invention provides a method of screening. The method includes contacting a candidate agent with a cellular array that includes a substrate comprising plurality of discrete sites and a plurality of cells dispersed at the discrete sites. Each cell is encoded with at least one exogenous binding partner. The method further includes determining the effect of the candidate agent on the cells.

In addition the invention provides a method of screening that includes contacting cells with an array that includes a substrate comprising a plurality of discrete sites and a plurality of microspheres dispersed at the sites, wherein each of the microspheres comprise a candidate bioactive agent.

### DETAILED DESCRIPTION OF THE PREFERRED EMBODIMENTS

In general, the invention provides for a biosensor, a biosensor array, a biosensor sensing system and sensing methods for the analysis of chemical and biological materials. More particularly, the invention provides for biosensors and biosensor arrays, sensing apparatus and sensing methods which employ living cells and mixed populations of living cells for analysis of chemical and biological materials.

The biosensor array of the present invention comprises either a monoculture of living cells or randomly mixed populations of living cells wherein each individual cell in the array is positioned on a substrate at an optically-addressable, discrete site which preferably accommodate the size and shape of individual cells. In one embodiment, the discrete site comprises a microwell or microcavity which is preformed to accommodate the size and shape of the individual cells. In some embodiments, as is more fully outlined below, the cells may be associated with microspheres that are loaded in the microwells. The biosensor array sensing method relies on the well known fact that individual cells, which are chemically or biologically stimulated by the presence of a biological or chemical material in the cell environment, will respond by producing a change in the cell or cellular environment which can be interrogated (generally optically) and detected within the cell itself or from an indicator compound, for example, a fluorophore, chromophore or dye, either attached to the cell, taken up in the cell, or added to the local cell environment. The biosensor of the present invention thus capitalizes on the ability of living cells to respond to biologically significant compounds. Since the selectivity of living cells for such compounds has considerable value and utility in candidate agent screening (e.g. drug screening) and analysis of complex biological fluids, the biosensor of the present invention offers distinct advantages to high throughput screening of combinatorial libraries where hundreds of thousands of candidate compounds must be evaluated.

By incorporating a biosensor into an optically interrogatable substrate or a fiber optic array, the innovation of the biosensor of the present invention is in providing for optical coupling of individual cells located at discrete substrate sites or microwells with discrete detector elements, CCD cameras, or individual optical fibers in a fiber optic array or bundle that are in optical communication with such devices. By “optical coupling”, “optical communication”, or “optical cooperation” or other grammatical equivalents herein is meant the capability of either optically stimulating individual cells within the biosensor array with excitation light or optically interrogating the optical response of individual cells within the array to analyses, by conveying light to and from individual cells located at discrete cites within the array using either conventional optical train elements or optical fibers. Since typical fiber optic arrays contain thousands of discrete individual fiber strands, the invention thus provides for the individual optical coupling and interrogation of thousands of cells within an array, thereby providing for a large number of independent cell response measurements for each cell population within an array. Due to both the number of cell populations available and the correspondingly large number of individual cells within each cell population, a significant innovation of the present invention is in providing for the summing and amplification of the characteristic optical response signatures of multiple independent measurements taken from cells within each cell population, thereby improving the detection limit and sensitivity of the biosensor.

An additional innovation of the present invention is that, by deploying a large number of cell populations within the array, and providing a large number of individual cells in each population, the discriminating capabilities of the biosensor array toward biological or chemical analyses is significantly enhanced by providing for thousands of cell responses from a large number of cell populations. This Feature directly mimics the actual behavior of the human olfactory system where the combined signals from thousands of receptor cells, in each grouping of nearly a thousand different receptor cell types found in the epithelium layer, none of which are particularly sensitive in themselves, lead to a highly amplified sensory response to odors [see Kauer, et al, Trehds Neurosci. 14: 79 (1991). One embodiment of the present invention thus mimics the evolutionary scent amplification process found in the human olfactory system in order to significantly enhance biosensor array sensitivity to analyses by summing the low-level responses of a large number of cells in the biosensor array. By summing the responses from a number of cells at low analyte concentrations, a substantial improvement in signal-to-noise ratio can be achieved and a corresponding reduction in the detection limit of the biosensor array is obtained.

Thus, the present invention provides cellular arrays. By “array” herein is meant a plurality of candidate agents in an array format; the size of the array will depend on the composition and end use of the array. Arrays containing from about 2 different bioactive agents (i.e. different beads) to many millions can be made, with very large fiber optic arrays being possible. Generally, the array will comprise from two to as many as a billion or more, depending on the size of the beads and the substrate, as well as the end use of the array, thus very high density, high density, moderate density, low density and very low density arrays may be made. Preferred ranges for very high density arrays are from about 10,000,000 to about 2,000,000,000, with from about 100,000,000 to about 1,000,000,000 being preferred (all numbers being in square cm). High density arrays range about 100,000 to about 10,000,000, with from about 1,000,000 to about 5,000,000 being particularly preferred. Moderate density arrays range from about 10,000 to about 100,000 being particularly preferred, and from about 20,000 to about 50,000 being especially preferred. Low density arrays are generally less than 10,000, with from about 1,000 to about 5,000 being preferred. Very low density arrays are less than 1,000, with from about 10 to about 1000 being preferred, and from about 100 to about 500 being particularly preferred. In some embodiments, the compositions of the invention may not be in array format; that is, for some embodiments, compositions comprising a single bioactive agent may be made as well. In addition, in some arrays, multiple substrates may be used, either of different or identical compositions. Thus for example, large arrays may comprise a plurality of smaller substrates.

In addition, one advantage of the present compositions is that particularly through the use of fiber optic technology, extremely high density arrays can be made. Thus for example, because beads of 200 μm or less (with beads of 200 nm possible) can be used, and very small fibers are known, it is possible to have as many as 250,000 or more (in some instances, 1 million) different fibers and beads in a 1 mm² fiber optic bundle, with densities of greater than 25,000,000 individual beads and fibers (again, in some instances as many as 50-100 million) per 0.5 cm² obtainable.

By “substrate” or “solid support” or other grammatical equivalents herein is meant any material that can be modified to contain discrete individual sites appropriate for the attachment or association of beads and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, Teflon, etc.), polysaccharides, nylon or nitrocellulose, resins, silica or silica-based materials including silicon and modified silicon, carbon, metals, inorganic glasses, plastics, optical fiber bundles, and a variety of other polymers. In general, the substrates allow optical detection and do not themselves appreciably fluoresces.

Generally the substrate is flat (planar), although as will be appreciated by those in the art, other configurations of substrates may be used as well; for example, three dimensional configurations can be used, for example by embedding the cells in a porous block of material such as plastic or agarose that allows solution access to the cells and using a confocal microscope for detection. Similarly, the cells may be placed on the inside surface of a tube, for flow-through sample analysis to minimize sample volume. Preferred substrates include optical fiber bundles as discussed below, and flat planar substrates such as glass, polystyrene and other plastics and acrylics.

In a preferred embodiment, the substrate is an optical fiber bundle or array, as is generally described in U.S. Ser. Nos. 08/944,850 and 08/519,062, PCT US98/05025, and PCT US98/09163, all of which are expressly incorporated herein by reference. Preferred embodiments utilize preformed unitary fiber optic arrays. By “preformed unitary fiber optic array” herein is meant an array of discrete individual fiber optic strands that are co-axially disposed and joined along their lengths. The fiber strands are generally individually clad. However, one thing that distinguished a preformed unitary array from other fiber optic formats is that the fibers are not individually physically manipulatable; that is, one strand generally cannot be physically separated at any point along its length from another fiber strand.

At least one surface of the substrate is modified to contain discrete, individual sites for later association of cells and/or microspheres. These sites may comprise physically altered sites, i.e. physical configurations such as wells or small depressions in the substrate that can retain the cells, such that a cell can rest in the well, or the use of other forces (magnetic or compressive), or chemically altered or active sites, such as chemically functionalized sites, electrostatically altered sites, hydrophobically/hydrophilically functionalized sites, spots of adhesive, etc.

The sites may be a pattern, i.e. a regular design or configuration, or randomly distributed. A preferred embodiment utilizes a regular pattern of sites such that the sites may be addressed in the X-Y coordinate plane. “Pattern” in this sense includes a repeating unit cell, preferably one that allows a high density of cells on the substrate. However, it should be noted that these sites may not be discrete sites. That is, it is possible to use a uniform surface of adhesive or chemical functionalities, for example, that allows the attachment of beads at any position. That is, the surface of the substrate is modified to allow attachment of the cells at individual sites, whether or not those sites are contiguous or non-contiguous with other sites. Thus, the surface of the substrate may be modified such that discrete sites are formed that can only have a single associated cell, or alternatively, the surface of the substrate is modified and cells may go down anywhere, but they end up at discrete sites.

In a preferred embodiment, the surface of the substrate is modified to contain wells, i.e. depressions in the surface of the substrate. This may be done as is generally known in the art using a variety of techniques, including, but not limited to, photolithography, stamping techniques, molding techniques and microetching techniques. As will be appreciated by those in the art, the technique used will depend on the composition and shape of the substrate.

In a preferred embodiment, physical alterations are made in a surface of the substrate to produce the sites. In a preferred embodiment, the substrate is a fiber optic bundle and the surface of the substrate is a terminal end of the fiber bundle, as is generally described in Ser. Nos. 08/818,199 and 09/151,877, both of which are hereby expressly incorporated by reference. In this embodiment, wells are made in a terminal or distal end of a fiber optic bundle comprising individual fibers. In this embodiment, the cores of the individual fibers are etched, with respect to the cladding, such that small wells or depressions are formed at one end of the fibers. The required depth of the wells will depend on the size of the beads to be added to the wells.

In a preferred embodiment, an array of micrometer-sized wells is created at the distal face of an optical imaging fiber by a selective etching process which takes advantage of the difference in etch rates between core and cladding materials. This process has been previously disclosed by Pantano, et al., Chem. Mater. 8: 2832 (1996), and Walt, et al., in U.S. patent application Ser. No. 08/818,199. The etch reaction time and conditions are adjusted to achieve desired control over the resultant microwell size and volume. Microwells are thus sized to accommodate a single cell of any desired cell type.

The sensor array design can accommodate a variety of cell sizes and configurations utilizing either commercially available optical fibers and fiber optic arrays or custom made fibers or fiber arrays. The major requirement in selecting candidate fibers or fiber optic arrays for fabricating sensors is that the individual fibers have etchable cores.

The method of fabricating microwells can be adapted to any fiber size so as to accommodate a wide range of cell sizes for incorporation into appropriately sized microwells. For example, optical fibers having core diameters ranging from 1.6 to 100 um are commercially available from either Galileo ElectroOptics Corp. (Sturbridge, Mass.) or Edmund Scientific (Barrington, N.J.). In addition, larger sizes are available by custom order. Thus, appropriately sized fibers can be utilized to study such diverse cell sizes as E. coli, with a typical cell dimension of 0.7 to 1.5 um, and mammalian neurons, with a cell dimension of up to 150 um.

It is generally preferred that the cells are non-covalently associated in the wells, although the wells may additionally be chemically functionalized as is generally described below, cross-linking agents may be used, or a physical barrier may be used, i.e. a film or membrane over the cells.

In a preferred embodiment, the surface of the substrate is modified to contain chemically modified sites, that can be used to attach, either covalently or non-covalently, either cells or microspheres of the invention to the discrete sites or locations on the substrate; however, in general, for covalent attachment, preferred embodiments utilize cells associated with microspheres that are chemically modified; that is, direct covalent attachment of cells to surfaces is generally not preferred although it can be done. “Chemically modified sites” in this context includes, but is not limited to, the addition of a pattern of chemical functional groups including amino groups, carboxy groups, oxo groups and thiol groups, that can be used to for covalent attachment, which generally also contain corresponding reactive functional groups; the addition of a pattern of adhesive that can be used to bind the microspheres (either by prior chemical functionalization for the addition of the adhesive or direct addition of the adhesive); the addition of a pattern of charged groups (similar to the chemical functionalities) for the electrostatic attachment of the cells or microspheres, e.g. when the microspheres comprise charged groups opposite to the sites; the addition of a pattern of chemical functional groups that renders the sites differentially hydrophobic or hydrophilic, such that the addition of similarly hydrophobic or hydrophilic microspheres under suitable experimental conditions will result in association of the microspheres to the sites on the basis of hydroaffinity. For example, the use of hydrophobic sites with hydrophobic beads, in an aqueous system, drives the association of the beads preferentially onto the sites. As outlined above, “pattern” in this sense includes the use of a uniform treatment of the surface to allow attachment of the beads at discrete sites, as well as treatment of the surface resulting in discrete sites. As will be appreciated by those in the art, this may be accomplished in a variety of ways.

In a preferred embodiment, biological modifications can be done to the sites to allow association of either cells or beads with cells. These include, but are not limited to, the use of binding ligands or binding partner pairs, including, but not limited to, antigen/antibody pairs, enzyme/substrate or inhibitor pairs, receptor-ligand pairs, carbohydrates and their binding partners (lectins, etc.).

In one embodiment, the interior surfaces of the microwells may be coated with a thin film or passivation layer of biologically compatible material, similar to the biological modifications of the substrate as outlined above. For example, materials known to support cell growth or adhesion may be used, including, but not limited to, fibronectin, any number of known polymers including collagen, polylysine and other polyamino acids, polyethylene glycol and polystyrene, growth factors, hormones, cytokines, etc. Preferred embodiments utilize cellular adhesion factors and proteins. Similarly, binding ligands as outlined above may be coated onto the surface of the wells. In addition, coatings or films of metals such as a metal such as gold, platinum or palladium may be employed. In an alternative embodiment, an indicator compound, for example, a fluorophore, a chromophore or dye, may be attached to the microwell surface for detecting cell responses to chemical or biological stimulation.

The cellular array comprises individual sensor elements comprising cells and/or beads. The arrays may comprise one type of cell or a plurality of different cell types. By “plurality” herein is meant at least two. As will be appreciated by those in the art, virtually any cell type and size can be accommodated in fabricating the sensor of the present invention; when wells in the ends of fiber optic bundles are used, the cell size may be matched to the individual optical fiber optic core diameters. Virtually any naturally occurring or genetically engineered (i.e. containing exogenous nucleic acid) eukaryotic or procaryotic cell type may be used, with plants, invertebrates, bacteria and mammalian cells, including, but not limited to, primate, rodent and human cells and cell lines being preferred, as well as mixtures of cell types.

In one embodiment, NIH 3T3 mouse fibroblast cells are employed. These cells are typically 15-20 um in size. Other cells types such as E. coli bacteria, 1×3 um, staphylococcus bacteria, approximately 1 um, myoblast precursors to skeletal muscle cells, 15-20 um, neutrophil white blood cells, 10 um, lymphocyte white blood cells, 10 um, erythroblast red blood cells, 5 um, osteoblast bone cells, 15-20 um, chondrocyte cartilage cells, 15-20 um, basophil white blood cells, 10 um, eosinophil white blood cells, 10 um, adipocyte fat cells, 20 um, invertebrate neurons (Helix aspera), 125 um, mammalian neurons, 4-140 um, or adrenomedullary cells, 13-16 um, melanocytes, 20 um, epithelial cells, 20 um, or endothelial cells, 15-20 um, may be utilized as well. Additional other suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T-cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. A particularly useful source of cell lines may be found in ATCC Cell Lines and Hybridomas (8^{th} ea., 1994), Bacteria and Bacteriophages (19^{th} ed., 1996), Yeast (1995), Mycology and Botany (19^{th}. ed, 1996), and Protists: Algae and Protozoa (18^{th} ed., 1993), available from American Type Culture Co. (Rockville, Md.), all of which are herein incorporated by reference.

Cellular arrays comprising cells may be made by randomly dispersal of the cells into or onto the sites of the array. Cell populations are conventionally cultured with growth media which matches cell needs. Culture media is formulated according to either recipes provided by cell line providers, journal articles or reference texts. A particularly useful reference for media preparation is ATCC Quality Control/Methods for Cell Lines (2nd ed.), American Type Culture Co. (Rockville, Md.) which is herein incorporated by reference. After culturing, cells are typically trypsinized using aseptic techniques to remove them from the cell culture dish and suspend them in growth media.

There are a variety of methods that can be employed to “load” the arrays. A preferred embodiment utilizes a cell suspension. In a preferred embodiment, prior to loading, the array surface may be sonicated under vacuum in cell media for approximately 15 minutes to flush and fill the microwells with media. The cell suspension is contacted with the substrate and allowed to interact for some period of time to allow the suspended cells to settle into the wells and adhere to the well bottom. The length of time required to fill the microwells is dependent only on the amount of time required for the cells to adhere to the microwell bottom. Excess cells which were are accommodated by a well can be removed.

Once they are positioned within the microwells, cells will typically attach to microwell surfaces within 1-2 hours by protein contact. Cell culture media in the microwells may be periodically replenished by exposing the array to fresh media and allowing nutrients to diffuse into the microwell cavities. Typically, cells will divide every twelve to fifteen hours. While the size of the microwells tends to confine individual cells, the array will accommodate limited cell splitting over time. Microwell volume will restrict cell splitting due to the well know cell phenomenon of contact inhibition when cells are touching.

In a preferred embodiment, the cells are associated with microspheres. By “microspheres” or “beads” or “particles” or grammatical equivalents herein is meant small discrete particles. The composition of the beads will vary, depending on a variety of factors. Suitable bead compositions include those used in peptide, nucleic acid and organic moiety synthesis, including, but not limited to, plastics, ceramics, glass, polystyrene, methylstyrene, acrylic polymers, paramagnetic materials, thoria sol, carbon graphite, titanium dioxide, agarose, latex or cross-linked dextrans such as Sepharose, cellulose, nylon, cross-linked micelles and Teflon may all be used. “Microsphere Detection Guide” from Bangs Laboratories, Fishers, Ind. is a helpful guide.

The beads need not be spherical; irregular particles may be used. In addition, the beads may be porous, thus increasing the surface area of the bead available for either binding partner attachment or cell association. The bead sizes range from nanometers, i.e. 100 nm, to millimeters, i.e. 1 mm, with beads of at least m150 micron being preferred, and from about 0.2 micron to about 200 microns being more preferred, and from about 0.5 to about 5 micron being particularly preferred, although in some embodiments smaller beads may be used.

It should be noted that a key component of the invention is the use of a substrate/bead pairing that allows the association or attachment of the beads at discrete sites on the surface of the substrate, such that the beads do not move during the course of the assay.

In a preferred embodiment, porous beads are used, such that the cells may infuse or be trapped within the beads, although care should be taken to allow media access to the cells. Alternatively, cells may be grown on the exposed surfaces of beads, including porous beads, using techniques known in the art for planar substrates.

Similarly, cells may be embedded in a bead matrix such as agarose, and microparticles generated with the resulting material.

In one embodiment, the cells are associated with the beads as outlined above for chemical or biological functionalization.

When cells are associated with microspheres, the cellular arrays can be fabricated as previously described for microsphere arrays; see PCT US98/05025 for example, hereby expressly incorporated by reference. In general, the arrays are made by adding a solution or slurry comprising the cell-bead compositions (sometimes referred to herein as “particles”) to a surface containing the sites for association of the particles. This may be done in a variety of buffers, including aqueous and organic solvents, and mixtures. The solvent can evaporate, and excess particles removed.

In a preferred embodiment, when non-covalent methods are used to associate the particles on the array, a novel method of loading the particles onto the array is used. This method comprises exposing the array to a solution of particles and then applying energy, e.g. agitating or vibrating the mixture. This results in an array comprising more tightly associated particles, as the agitation is done with sufficient energy to cause weakly-associated particles to fall off (or out, in the case of wells). These sites are then available to bind a different particle. In this way, particles that exhibit a high affinity for the sites are selected. Arrays made in this way have two main advantages as compared to a more static loading: first of all, a higher percentage of the sites can be filled easily, and secondly, the arrays thus loaded show a substantial decrease in particle loss during assays. Thus, in a preferred embodiment, these methods are used to generate arrays that have at least about 50% of the sites filled, with at least about 75% being preferred, and at least about 90% being particularly preferred. Similarly, arrays generated in this manner preferably lose less than about 20% of the particles during an assay, with less than about 10% being preferred and less than about 5% being particularly preferred.

In this embodiment, the substrate comprising the surface with the discrete sites is immersed into a solution comprising the particles (beads, cells, etc.). The surface may comprise wells, as is described herein, or other types of sites on a patterned surface such that there is a differential affinity for the sites. This differential affinity results in a competitive process, such that particles that will associate more tightly are selected. Preferably, the entire surface to be “loaded” with particles is in fluid contact with the solution. This solution is generally a slurry ranging from about 10,000:1 beads:solution (vol:vol) to 1:1. Generally, the solution can comprise any number of reagents, including aqueous buffers, organic solvents, salts, other reagent components, etc. In addition, the solution preferably comprises an excess of particles; that is, there are more particles than sites on the array. Preferred embodiments utilize two-fold to billion-fold excess of particles.

The immersion can mimic the assay conditions; for example, if the array is to be “dipped” from above into a microtiter plate comprising samples, this configuration can be repeated for the loading, thus minimizing the particles that are likely to fall out due to gravity.

Once the surface has been immersed, the substrate, the solution, or both are subjected to a competitive process, whereby the particles with lower affinity can be disassociated from the substrate and replaced by particles exhibiting a higher affinity to the site. This competitive process is done by the introduction of energy, in the form of heat, sonication, stirring or mixing, vibrating or agitating the solution or substrate, or both. Care should be taken to avoid damaging the cells.

A preferred embodiment utilizes agitation or vibration. In general, the amount of manipulation of the substrate is minimized to prevent damage to the array; thus, preferred embodiments utilize the agitation of the solution rather than the array, although either will work. As will be appreciated by those in the art, this agitation can take on any number of forms, with a preferred embodiment utilizing microtiter plates comprising bead solutions being agitated using microtiter plate shakers.

The agitation proceeds for a period of time sufficient to load the array to a desired fill. Depending on the size and concentration of the particles and the size of the array, this time may range from about 1 second to days, with from about 1 minute to about 24 hours being preferred.

It should be noted that not all sites of an array may comprise a cell; that is, there may be some sites on the substrate surface which are empty. In addition, there may be some sites that contain more than one cell. This is acceptable if for example beads are used and only one bead, that may comprise a plurality of cells of a single cell type, is associated with the site.

In addition, cell viability tests such as are known in the art may be done after loading of the arrays. These include, but are not limited to, the use of pH indicators and well known dyes for cell viability; see the Molecular Probes Handbook, supra.

In contrast to the systems described in U.S. Ser. Nos. 09/033,462 and 09/260,963, the present invention provides for novel and robust methods of encoding the individual cells and cell populations in the array for maintaining cell type identity and location where randomly mixed populations of cells are employed. Cells may be encoded prior to disposing them in the microwells (or at the sites) or, alternatively, following placement in the microwells. Although cell populations may be randomly mixed together, this innovative feature provides for the identity and location of each cell type to be determined via a characteristic optical response signature when the cell array is either illuminated by excitation light energy or, alternatively, subjected to biological stimuli.

The invention provides for novel encoding methods, including, but not limited to, the use of identifier binding ligands (IBLs), preferably that bind to antibodies or fluorophores. That is, by using unique ligands that will bind a unique partner, the identity of the cell type may be elucidated. For example, cell surface receptors specific to immunological cells can be used with uniquely labeled ligands to distinguish from cells not carrying the receptor; upon binding of the ligand to the receptor, the label is now present at the array site and can be detected; antibodies to cell surface receptors will also work well. The IBLs may be either endogenous (native to the cell) or exogenous (not native to the cell, e.g. recombinantly introduced).

By “identifier binding ligands” or “IBLs” herein is meant a compound that will specifically bind a corresponding decoder binding ligand (DBL) to facilitate the elucidation of the identity of the cell. That is, the IBL and the corresponding DBL form a binding partner pair. By “specifically bind” herein is meant that the IBL binds its DBL with specificity sufficient to differentiate between the corresponding DBL and other DBLs (that is, DBLs for other IBLs), or other components or contaminants of the system. The binding should be sufficient to remain bound under the conditions of the decoding step, including wash steps to remove non-specific binding. In some embodiments, for example when the IBLs and corresponding DBLs are proteins or nucleic acids, the dissociation constants of the IBL to its DBL will be less than about 10⁻⁴-10⁻⁶ M⁻¹, with less than about 10⁻⁵ to 10⁻⁹ M⁻¹ being preferred and less than about 10⁻⁷-10⁻⁹ M⁻¹ being particularly preferred.

IBL-DBL binding pairs are known or can be readily found using known techniques. For example, when the IBL is a protein, the DBLs include proteins (particularly including antibodies or fragments thereof (FAbs, etc.)) or small molecules, or vice versa (the IBL is an antibody and the DBL is a protein). Metal ion-metal ion ligands or chelators pairs are also useful. Antigen-antibody pairs, enzymes and substrates or inhibitors, other protein-protein interacting pairs, receptor-ligands, complementary nucleic acids (including nucleic acid molecules that form triple helices), and carbohydrates and their binding partners are also suitable binding pairs. Nucleic acid—nucleic acid binding proteins pairs are also useful, including single-stranded or double-stranded nucleic acid binding proteins, and small molecule nucleic acid binding agents. Similarly, as is generally described in U.S. Pat. Nos. 5,270,163, 5,475,096, 5,567,588, 5,595,877, 5,637,459, 5,683,867,5,705,337, and related patents, hereby incorporated by reference, nucleic acid “aptamers” can be developed for binding to virtually any target; such an aptamer-target pair can be used as the IBL-DBL pair. Similarly, there is a wide body of literature relating to the development of binding pairs based on combinatorial chemistry methods.

In a preferred embodiment, each subpopulation of cells (e.g. each cell type) comprises a plurality of different IBLs. By using a plurality of different IBLs to encode each cell, the number of possible unique codes is substantially increased. That is, by using one unique IBL per cell, the size of the array will be the number of unique IBLs (assuming no “reuse” occurs, as outlined below). That is, 20 different cell types can be distinguished (assuming sufficient specificity and thus a lack of cross-reactivity) using 20 different IBLs. However, by using a plurality of different IBLs per cell, n, the size of the array can be increased to 2ⁿ, when the presence or absence of each IBL is used as the indicator. For example, the assignment of 10 IBLs per bead generates a 10 bit binary code, where each bit can be designated as “1” (IBL is present) or “0” (IBL is absent). A 10 bit binary code has 2¹⁰ possible variants. However, as is more fully discussed below, the size of the array may be further increased if another parameter is included such as concentration or intensity; thus for example, if two different concentrations of the IBL are used (e.g. two different expression levels), then the array size increases as 3ⁿ. Thus, in this embodiment, each individual cell in the array is assigned a combination of IBLs.

In addition, the use of different concentrations or densities of IBLs allows a “reuse” of sorts. If, for example, a first cell type comprises an expression vector that results in a 1× expression level of IBL, and a second cell type comprises a different construct that gives a 10× expression level of IBL, using saturating concentrations of the corresponding labeled DBL allows the user to distinguish between the two cell types.

As outlined herein, the IBL-DBL pair may be selected from any number of binding partner pairs, with antigen-antibody pairs, other cell surface receptor-ligand pairs (including for example, insulin receptor, insulin-like growth factor receptor, growth hormone receptor, glucose transporters (particularly GLUT 4 receptor), transferrin receptor, epidermal growth factor receptor, low density lipoprotein receptor, high density lipoprotein receptor, epidermal growth factor receptor, leptin receptor, interleukin receptors including IL-1, IL-2, IL-3, IL-4, IL-5, IL-6, IL-7, IL-8, IL-9, IL-11, IL-12, IL-13, IL-15, and IL-17 receptors, human growth hormone receptor, VEGF receptor, PDGF receptor, EPO receptor, TPO receptor, ciliary neurotrophic factor receptor, prolactin receptor, and T-cell receptors; all of which have associated ligands), and fluorophore binding peptides as is known in the art; see Rozinov Chem. Biol. 5: 713 (1998) and Whitney et al., Nature Biotech. 16: 1329 (1998), both of which are expressly incorporated by reference.

In a preferred embodiment, the IBL is expressed on the surface of the cell, to allow access of binding partners such as antibodies or fluorophores. This is done as is generally known in the art, using specific signal sequences or membrane anchoring sequences, including but not limited to, Particularly preferred membrane-anchoring sequences include, but are not limited to, those derived from CD8, ICAM-2, IL-8R, CD4 and LFA-1; the GPI anchor, which results in a covalent bond between the molecule and the lipid bilayer via a glycosyl-phosphatidylinositol bond; myristylation sequences, which serve as membrane anchoring sequences since the myristylation of c-src recruits it to the plasma membrane; and palmitoylation sequences.

However, in some embodiments the IBLs are located within the cell, and the DBLs are introduced into the cell as well.

In general, cells are transformed using techniques well known in the art. Preferred embodiments utilize a variety of vectors and constructs with phage vectors being particularly preferred, including, but not limited to, retroviruses, adenoviruses, FIV, lentiviruses, etc.

When IBLs (e.g. codes) are introduced to cells by way of recombinant techniques, preferred embodiments utilize the delivery of additional nucleic acids, which can serve either as candidate agents, as outlined herein, or for more specific purposes. For example, the additional nucleic acid could be a new gene added to a cell, or an antisense gene. Linking new nucleic acid with a “code” allows the tracking of changes within the cell; this tracking can create a functional assay for determining the effectors of a gene (including non-coding regions) when introduced to a cell.

In a preferred embodiment, when cells are first associated with beads to form “particles” as outlined herein, the IBLs may be found on the beads. That is, subpopulations of cell types are associated with beads that comprise IBLs that can be used to locate these beads.

In a preferred embodiment, the location of the different cell types on the array is determined using decoder binding ligands (DBLs). As outlined above, DBLs are binding ligands that will bind to identifier binding ligands.

In a preferred embodiment, the DBLs are either directly or indirectly labeled. By “labeled” herein is meant that a compound has at least one element, isotope or chemical compound attached to enable the detection of the compound. In general, labels fall into three classes: a) isotopic labels, which may be radioactive or heavy isotopes; b) magnetic, electrical, thermal; and c) colored or luminescent dyes; although labels include enzymes and particles such as magnetic particles as well. Preferred labels include luminescent labels. In a preferred embodiment, the DBL is directly labeled, that is, the DBL comprises a label. In an alternate embodiment, the DBL is indirectly labeled; that is, a labeling binding ligand (LBL) that will bind to the DBL is used. In this embodiment, the labeling binding ligand-DBL pair can be as described above for IBL-DBL pairs. Suitable labels include, but are not limited to, fluorescent lanthanide complexes, including those of Europium and Terbium, fluorescein, rhodamine, tetramethylrhodamine, eosin, erythrosin, coumarin, methyl-coumarins, pyrene, Malacite green, stilbene, Lucifer Yellow, Cascade Blue, Texas Red, FITC, PE, cy3, cy5 and others described in the 6th Edition of the Molecular Probes Handbook by Richard P. Haugland, hereby expressly incorporated by reference.

Accordingly, the identification of the location of the individual cells (or subpopulations of cells) is done using one or more decoding steps comprising a binding between the labeled DBL and the IBL. After decoding, the DBLs can be removed and the array can be used; however, in some circumstances, the removal of the DBL is not required (although it may be desirable in some circumstances). In addition, as outlined herein, decoding may be done either before the array is used in an assay, during the assay, or after the assay.

In one embodiment, a single decoding step is done. In this embodiment, each DBL is labeled with a unique label, such that the number of unique labels is equal to or greater than the number of different cell types (although in some cases, “reuse” of the unique labels can be done, as described herein). For each IBL, a DBL is made that will specifically bind to it and contains a unique label, for example one or more fluorochromes. Thus, the identity of each DBL, both its composition and its label, is known. Then, by adding the DBLs to the array containing the cells under conditions which allow the formation of binding complexes between the DBLs and the IBLs, the location of each DBL can be elucidated. This allows the identification of the location of each cells; the random array has been decoded. The DBLs can then be removed, if necessary, and the target sample applied.

In a preferred embodiment, the number of unique labels is less than the number of unique cell types, and thus a sequential series of decoding steps are used, as is outlined in U.S. Ser. Nos. 09/189,543 and 09/344,526, hereby incorporated by reference.

In one embodiment, the DBLs are labeled in situ; that is, they need not be labeled prior to the decoding reaction.

In a preferred embodiment, the DBLs may be reused by having some subpopulations of cells comprise optical signatures as is generally described in U.S. Ser. Nos. 09/033,462 and 09/260,963. In a preferred embodiment, the optical signature is generally a mixture of reporter dyes, preferably flourescent. By varying both the composition of the mixture (i.e. the ratio of one dye to another) and the concentration of the dye (leading to differences in signal intensity), matrices of unique optical signatures may be generated.

Once made and decoded as required, the cellular arrays of the present invention find use in a number of applications. The biosensor, biosensor array, sensing apparatus and sensing method of the present invention can be applied to a large variety of conventional assays for screening and detection purposes. The biosensor may be configured for virtually any assay and offers a distinct advantage for high throughput screening where a plurality of encoded cell populations, which have utility in particular assays or are genetically engineered cell to provide unique responses to analytes, may be employed in a single sensor array for conducting a large number of assays simultaneously on a small sample. The biosensor array thus provides both for tremendous efficiencies in screening large combinatorial libraries and allows conduction of a large number of assays on extremely small sample volumes, such as biologically important molecules synthesized on micron sized beads. The biosensor of the present invention can be applied to virtually any analyte measurements where there is a detectable cell response to the analyte due to biological stimulation.

The biosensor array and method of the present invention utilizes the unique ability of living cell populations to respond to biologically significant compounds in a characteristic and detectable manner. Since the selectivity of living cells for such compounds has considerable value and utility in drug screening and analysis of complex biological fluids, a biosensor which makes use of the unique characteristics of living cell populations offers distinct advantages in high throughput screening of combinatorial libraries where hundreds of thousands of candidate pharmaceutical compounds must be evaluated. In addition, such a biosensor and sensing method can be utilized for either off-line monitoring of bioprocesses or in situ monitoring of environmental pollutants where the enhanced sensitivity of living cells to their local environment can be exploited.

Thus, the present invention provides methods for detecting the responses of individual cells to analyses of interest. By “analyte of interest” or “target analyte” or “candidate bioactive agent” or “candidate drug” or grammatical equivalents herein is meant any molecule, e.g., protein, oligopeptide, small organic molecule, polysaccharide, polynucleotide, etc., to be tested for the ability to directly or indirectly altering a cellular phenotype, including its optical properties. Generally a plurality of assay mixtures are run in parallel with different agent concentrations to obtain a differential response to the various concentrations. Typically, one of these concentrations serves as a negative control, i.e., at zero concentration or below the level of detection.

Analytes encompass numerous chemical classes, though typically they are organic molecules, preferably small organic compounds having a molecular weight of more than 100 and less than about 2,500 daltons. Analytes comprise functional groups necessary for structural interaction with proteins, particularly hydrogen bonding, and typically include at least an amine, carbonyl, hydroxyl or carboxyl group, preferably at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. Particularly preferred are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In a preferred embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. Particularly preferred in this embodiment are libraries of bacterial, fungal, viral, and mammalian proteins, with the latter being preferred, and human proteins being especially preferred.

The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In a preferred embodiment, the library is biased. That is, some positions within the sequence are either held constant, or are selected from a limited number of possibilities. For example, in a preferred embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross-linking, prolines for SH-3 domains, serines, threonines, tyrosines or histidines for phosphorylation sites, etc., or to purines, etc.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins. By “nucleic acid” or “oligonucleotide” or grammatical equivalents herein means at least two nucleotides covalently linked together. A nucleic acid of the present invention will generally contain phosphodiester bonds, although in some cases, as outlined below, nucleic acid analogs are included that may have alternate backbones, comprising, for example, phosphoramide (Beaucage et al., Tetrahedron 49(10): 1925 (1993) and references therein; Letsinger, J. Org. Chem. 35: 3800 (1970); Sprinzl et al., Eur. J. Biochem. 81: 579 (1977); Letsinger et al., Nucl. Acids Res. 14: 3487 (1986); Sawai et al, Chem. Lett. 805 (1984), Letsinger et al., J. Am. Chem. Soc. 110: 4470 (1988); and Pauwels et al., Chemica Scripta 26: 141 91986)), phosphorothioate (Mag et al., Nucleic Acids Res. 19: 1437 (1991); and U.S. Pat. No. 5,644,048), phosphorodithioate (Briu et al., J. Am. Chem. Soc. 111: 2321 (1989), O-methylphophoroamidite linkages (see Eckstein, Oligonucleotides and Analogues: A Practical Approach, Oxford University Press), and peptide nucleic acid backbones and linkages (see Egholm, J. Am. Chem. Soc. 114: 1895 (1992); Meier et al., Chem. Int. Ed. Engl. 31: 1008 (1992); Nielsen, Nature, 365: 566 (1993); Carlsson et al., Nature 380: 207 (1996), all of which are incorporated by reference). Other analog nucleic acids include those with positive backbones (Denpcy et al., Proc. Natl. Acad. Sci. USA 92: 6097 (1995); non-ionic backbones (U.S. Pat. Nos. 5,386,023, 5,637,684, 5,602,240, 5,216,141 and 4,469,863; Kiedrowshi et al., Angew. Chem. Intl. Ed. English 30: 423 (1991); Letsinger et al., J. Am. Chem. Soc. 110: 4470 (1988); Letsinger et al., Nucleoside & Nucleotide 13: 1597 (1994); Chapters 2 and 3, ASC Symposium Series 580, “Carbohydrate Modifications in Antisense Research”, Ed. Y. S. Sanghui and P. Dan Cook; Mesmaeker et al., Bioorganic & Medicinal Chem. Lett. 4: 395 (1994); Jeffs et al., J. Biomolecular NMR 34: 17 (1994); Tetrahedron Lett. 37: 743 (1996)) and non-ribose backbones, including those described in U.S. Pat. Nos. 5,235,033 and 5,034,506, and Chapters 6 and 7, ASC Symposium Series 580, “Carbohydrate Modifications in Antisense Research”, Ed. Y. S. Sanghui and P. Dan Cook. Nucleic acids containing one or more carbocyclic sugars are also included within the definition of nucleic acids (see Jenkins et al., Chem. Soc. Rev. (1995) pp 169-176). Several nucleic acid analogs are described in Rawls, C & E News Jun. 2, 1997 page 35. All of these references are hereby expressly incorporated by reference. These modifications of the ribose-phosphate backbone may be done to facilitate the addition of labels, or to increase the stability and half-life of such molecules in physiological environments.

As will be appreciated by those in the art, all of these nucleic acid analogs may find use in the present invention. In addition, mixtures of naturally occurring nucleic acids and analogs can be made; alternatively, mixtures of different nucleic acid analogs, and mixtures of naturally occurring nucleic acids and analogs may be made.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo- and ribo-nucleotides, and any combination of bases, including uracil, adenine, thymine, cytosine, guanine, inosine, xathanine hypoxathanine, isocytosine, isoguanine, etc. As used herein, the term “nucleoside” includes nucleotides as well as nucleoside and nucleotide analogs, and modified nucleosides such as amino modified nucleosides. In addition, “nucleoside” includes non-naturally occurring analog structures. Thus for example the individual units of a peptide nucleic acid, each containing a base, are referred to herein as a nucleoside.

In a preferred embodiment, the candidate bioactive agents are organic chemical moieties, a wide variety of which are available in the literature.

While the examples below provide a variety of specific assays which may be useful in configuring and employing the biosensor array and method of the present invention, they are not intended to limit either the scope of applications envisioned or the broad range of sensing methods which can be employed with a plurality of cell populations with the biosensor of the present invention.

In one embodiment, the biosensor array can be employed for remotely monitoring redox states of individual cells or cell populations in bioprocesses. For example, NADH dependent fluorescence can be measured in bacteria, fungi, plant or animal cells. NAD(P)/NAD(P)H can be measured to monitor changes from aerobic to anaerobic metabolism in fermentation processes using the method disclosed by Luong, et al., in Practical Fluorescence, G. Guilbault ed., Marcel Dekker (New York, 1990).

Alternatively, the biosensor array may be employed for in situ monitoring of cellular processes in response to environmental contaminants by incorporating the method disclosed by Hughes, et al., Analytica Chimica Acta 307: 393 (1995) to provide for distinguishable cell population responses within an array. In this method, micron-sized spheres, impregnated with a fluorophore and modified on the surface with a fluorogenic enzyme probe, are ingested by cells and enzymatic activity occurs at the sphere surface, producing a detectable fluorescent signal.

In yet another embodiment, the biosensor array can be employed with genetically engineered bioluminescent bacteria for in situ monitoring and optical sensing of metallic compounds. For example, cell population responses to antimonite and arsenite may be utilized by incorporating the method disclosed in Ramanathan, et al., Anal Chem. 69: 3380 (1997) into cell populations within the biosensor array. With this method, cell plasmid regulates the expression of bacterial luciferase depending on the metal concentration.

In another embodiment, the cell populations within the biosensor array can be encoded with ATP dependent luminescent proteins, for example firefly luciferase, which are injected into rat hepatocytes for pathological studies according to the method disclosed by Koop, et al., Biochem. J. 295: 165 (1993). These cells exhibit a decrease in cytoplasmic ATP when exposed to pathological insults and changes in fluorescence directly relate to the extent of metabolic poisoning in the cell.

In one embodiment, the cell populations within the biosensor array can be encoded with green fluorescent protein [see T. Gura, Science 276: 1989 (1997); Niswender, et al., J. Microscopy 180(2): 109 (1995); Cubitt, et al., TIBS 20: 448 (1995); Miyawaki, et al., Nature 388: 882 (1997)]. Several genetically-engineered mutants of GFP are available which have distinguishable fluorescence emission wavelengths. These proteins have additional utility as fluorescing indicators of gene expression and Ca⁺ levels within cells.

In an additional embodiment, the biosensor array can be used in measurements of cell proliferation by in situ monitoring of calcium levels and calcium oscillations in single cells using fluorescent markers, such as aequorin or fura-2, according to the method disclosed by Cobbold, et al., Cell Biology 1: 311 (1990).

As will be appreciated by those in the art, the assays of the invention may be run in a wide variety of ways and for a wide variety of purposes. For example, the cells may be used as a detection system for a particular analyte; the cells undergo a characteristic optically detectable change in the presence of a particular analyte. Alternatively, the cells may be used to screen drug candidate libraries for the ability to alter a cellular phenotype that is optically detectable. For example, the expression of a therapeutically relevant cell surface receptor may be increased such that the receptor can now bind a fluorescent ligand; similarly a therapeutically relevant enzyme may now be activated such that a fluorescent reaction product is generated. Alternatively, the candidate agent(s) may be introduced, and then a secondary marker is added to detect changes in cellular states; for example, apoptosis can be detected using fluorescent annexin, etc. See the Molecular Probes Handbook, supra. In this way any modulation, including both increases and decreases, may be monitored. Similarly, the use of reporter genes such as green fluorescent proteins and derivatives thereof facilitates high throughput screening for relevant analyte interactions, through the use of inducible promoters, for example.

Generally, in a preferred embodiment, a candidate bioactive agent is added to the cells prior to analysis, and the cells allowed to incubate for some period of time. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, the cells can be washed if desired and are allowed to incubate under preferably physiological conditions for some period of time.

The reactions outlined herein may be accomplished in a variety of ways, as will be appreciated by those in the art. Components of the reaction may be added simultaneously, or sequentially, in any order. In addition, the reaction may include a variety of other reagents may be included in the assays. These include reagents like salts, buffers, neutral proteins, e.g. albumin, detergents, etc which may be used to facilitate optimal detection, and/or reduce non-specific or background interactions. Also reagents that otherwise improve the efficiency of the assay, such as protease inhibitors, nuclease inhibitors, antimicrobial agents, etc., may be used.

In general, at least one component of the assay is labeled. By “labeled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc. For the specific binding members, the complementary member would normally be labeled with a molecule which provides for detection, in accordance with known procedures, as outlined above. The label can directly or indirectly provide a detectable signal.

Once the assay is run, the data is analyzed to determine the experimental outcome, i.e. either the presence or absence of a target analyte, the effect of a candidate agent on a cellular phenotype, etc. This is generally done using a computer.

In this way, bioactive agents are identified. Compounds with pharmacological activity are able to alter a cellular phenotype. The compounds having the desired pharmacological activity may be administered in a physiologically acceptable carrier to a host, as previously described, he agents may be administered in a variety of ways, orally, parenterally e.g., subcutaneously, intraperitoneally, intravascularly, etc. Depending upon the manner of introduction, the compounds may be formulated in a variety of ways. The concentration of therapeutically active compound in the formulation may vary from about 0.1-100 wt. %.

The pharmaceutical compositions can be prepared in various forms, such as granules, tablets, pills, suppositories, capsules, suspensions, salves, lotions and the like. Pharmaceutical grade organic or inorganic carriers and/or diluents suitable for oral and topical use can be used to make up compositions containing the therapeutically-active compounds. Diluents known to the art include aqueous media, vegetable and animal oils and fats. Stabilizing agents, wetting and emulsifying agents, salts for varying the osmotic pressure or buffers for securing an adequate pH value, and skin penetration enhancers can be used as auxiliary agents.

Accordingly, individual cells or arrays of cells and cell populations may be optically interrogated and cell responses to analyses may be measured by conventional optical methods and instrumentation that are known to those skilled in the art. Cells may be optically interrogated with any suitable excitation light energy source, such as arc lamps, lasers, or light emitting diodes, that are capable of producing light at an appropriate wavelength for exciting dye indicators that may be employed for encoding cell populations or for responding to analyses of interest. The optical responses of individual cells or cell populations may be monitored and measured with any suitable optical detection means, including, but not limited to film or conventional optical detectors, such as photoresistors, photomultiplier tubes, photodiodes, or charge coupled device (CCD) cameras. In a preferred embodiment, CCD cameras are employed to capture fluorescent images of the biosensor array for detecting responses of each cell and various cell subpopulations to analyses. Where external optical stimulation of cells is required to elicit an optical response, conventional light sources such as arc lamps, photodiodes, or lasers may be employed for excitation light energy. Cell responses may be monitored by conventional detectors such as photomultiplier tubes, photodiodes, photoresistors or charge coupled device (CCD) cameras. Conventional optical train components, such as lenses, filters, beam splitters, dichroics, prisms and mirrors may be employed to convey light to an from such light sources and detectors either to discrete substrate sites or through optical fiber strands to microwells that contain individual cells. The principal requirement for any particular optical apparatus configuration that is employed in optical measurements is that the combination of optical components provide for optically coupling the cells in the array to detectors and light sources. While a particular apparatus configuration that was employed in experimental optical measurements is described below, other configurations may also be employed which are functionally equivalent and appropriate suited for a particular measurement requirement. In this embodiment, both individual cell responses and a captured image of the array response may be employed for detecting analytes.

In summary, the biosensor array and sensing method of the present invention offers many distinct advantages in overcoming the limitations of prior art devices. The sensor arrays are easily fabricated from commercially available optical imaging fibers to yield a cost effective, high density, precisely formed, biosensor array without requiring any sophisticated machining or forming process. Since optical fibers and fiber optic arrays are available in a wide variety of fiber core diameters, most cell types and sizes may be accommodated in by the device and method of the present invention. In addition, cells can be readily dispersed into the microwell array in random fashion with no need for physical indexing or scanning to locate individual cells or cell populations due to the innovative cell encoding technique. Sensing methods and sensing systems which employ the biosensor and sensor array of the present invention avoids many of the limitations in manipulating cells encountered with prior art devices. Once cells are placed within the microwells of the array, conventional imaging systems and methods which employ an imaging camera and conventional optics, can monitor the response of thousands of cells simultaneously, eliminating requirements for mechanical scanning mechanisms. Analysis of measurement data is further facilitated by implementing commercially available imaging software to process images of the biosensor array using pattern recognition techniques combined with neural network and other statistical methods.

The biosensor array and sensing method of the present invention may be employed for a number of useful analytical applications where individual cells, which are chemically or biologically stimulated by the presence of a biological or chemical material in the local cell environment, will respond to their environment by producing an optically detectable response either due to the presence of an appropriate indicator or due to the characteristic optical response of particular cell types which exhibit either natural or genetically-engineered chemiluminescence or bioluminescence. The biosensor array and method of the present invention thus capitalizes on the ability of living cells to respond to biologically significant compounds. Since the selectivity of living cells for such compounds has considerable value and utility in drug screening and analysis of complex biological fluids, the biosensor of the present invention offers distinct advantages to high throughput screening of combinatorial libraries where hundreds of thousands of candidate compounds must be evaluated.

The present invention also provides methods of using microsphere arrays for screening candidate agents on cells. In this embodiment, candidate agents (preferably libraries of candidate agents) are attached to beads on an array, preferably through a labile linker such as a photocleavable linker. That is, candidate bioactive agents are synthesized on a bead (which may be porous), resulting in the candidate agents being attached, frequently via a scissile linkage, to the surface (which can be both internal and external) of a bead. The beads are distributed at discrete sites on a surface as described herein for cells and microspheres, for example a terminus of a fiber optic bundle, such that extremely large numbers of assays can be simultaneously run. The candidate bioactive agent is optionally cleaved from the bead, allowing free diffusion through the bead and interaction with the target cell. Detection of binding or activity is then accomplished in a variety of ways, as is more fully outlined herein and known in the art.

Thus the present invention has a number of important advantages, including the use of very low amounts of reagents, a distinct spatial localization of the assays, decreased propensity to diffusion and decreased rates of solvent evaporation. In addition, as is more fully outlined below, by varying the amount of candidate bioactive agent attached to each bead, the concentration of the candidate bioactive agent can be varied, allowing the simultaneous determination of concentration effects. This is quite significant, as it allows the determination of kinetic parameters such as Kₘ and Kᵢ and also avoids subsequent scale-up, including resynthesis of the agents and reassays.

The cells are contacted with the beads, either as a component of the bead (i.e. the cells are in or on the bead as outlined above) or by contacting the surface of the array with a “lawn” of cells.

In a preferred embodiment, the cells and candidate agents are on beads. That is, the microspheres comprising the candidate agents are synthesized, and then cells are associated with the beads; again, as outlined above, this may be done in a variety of ways. For example, porous beads may be used and the cells either grown in the pores or entrapped in the pores. Similarly, when very porous substances such as agarose are used, the cells may be embedded in the beads. Alternatively, the cells may be associated with the external surface of the beads, similar to the manner that cells attach to planar surfaces. In this embodiment, it may not be necessary to cleave the candidate agents off the beads to see effects, particularly for agents that interact with cell surface moieties. However, cleavage of the agent off the bead can allow for diffusion into or onto a cell.

Alternatively, a preferred embodiment utilizes arrays comprising beads with candidate agents which is later contacted with a substrate comprising cells. That is, an microsphere array is made (and decoded, if necessary, although decoding may not occur until after the assay) and then contacted with cells, either as a “lawn” of cells that are grown on a substrate or tissue samples, etc. The bioactive agents are preferably cleaved off of the bead, for example using photolysis, and then diffuse either onto or into the cells.

Suitable scissile linkages are described in U.S. Ser. No. 60/119,343, hereby incorporated by reference in its entirety.

The above and other features of the invention, including various novel details of construction and methods, and other advantages, will now be more particularly described with reference to the accompanying claims. It will be understood to one skilled in the art that the particular apparatus and method embodying the invention are shown by way of illustration and not as a limitation of the invention. The principles and features of the invention may be employed in various and numerous embodiments without departing from the scope of the invention. All references cited herein are expressly incorporated by reference in their entirety.

1. A cellular array for detecting the response of individual cells to at least one analyte of interest comprising:
a) a substrate comprising a plurality of discrete sites; and
b) a plurality of cells dispersed at said discrete sites, wherein each cell is encoded with at least one exogenous binding partner.

2. A cellular array according to claim 1, wherein said substrate is a fiber optic bundle.

3. A cellular array according to claim 1 or 2, wherein said discrete sites comprise wells.

4. A cellular array according to claim 1, 2 or 3 wherein said exogenous binding partner is an antibody ligand.

5. A cellular array according to claim 1, 2, 3 or 4 wherein each cell is encoded with at least two exogenous binding partners.

6. A cellular array according to claim 1, 2, 3, 4 or 5 wherein said exogenous binding partner is a fluorophore binding peptide.

7. A cellular array according to claim 1, 2, 3, 4, 5 or 6 wherein said cells further comprise candidate agents.

8. A method of making a cellular array comprising:
a) associating a population of cells with a population of microspheres, such that each microsphere has at least one associated cell, wherein said population comprises a first subpopulation comprising a first cell type and at least a second subpopulation comprising a second cell type, wherein said first subpopulation comprises at least a first exogenous binding partner and said second subpopulation comprises at least a second exogenous binding partner;
b) distributing said population of microspheres onto discrete sites of a substrate; and
c) identifying the location of at least said first and said second exogenous binding partners.

9. A method according to claim 8 wherein said microspheres are porous and said cells are entrapped within said microspheres.

10. A method according to claim 8 wherein said cells are grown on the surface of said microspheres.

11. A method according to claim 8 wherein said microspheres comprise a first binding ligand and said cells comprise a binding partner.

12. A method of screening comprising:
a) contacting a candidate agent with a cellular array comprising:
i) a substrate comprising plurality of discrete sites; and
ii) a plurality of cells dispersed at said discrete sites, wherein each cell is encoded with at least one exogeneous binding partner; and
b) determining the effect of said candidate agent on said cells.

13. A method of screening comprising:
a) contacting cells with an array comprising:
i) a substrate comprising a plurality of discrete sites; and
ii) a plurality of microspheres dispersed at said sites, wherein each of said microspheres comprise a candidate bioactive agent.

14. A method according to claim 13 wherein said bioactive agents are linked to said microspheres via a photolabile linkage.

15. A method according to claim 14 wherein said method further comprises releasing said candidate bioactive agents.
